(19) United States
(12) Patent Application Publication
(10) Pub. No.: US 2012/0232402 A1
(43) Pub. Date: Sep. 13, 2012

(54) Functional Near Infrared Spectroscopy Imaging System and Method

(76) Inventors: Duncan MacFarlane, Dallas, TX (US); Chester Wildey, Euless, TX (US); Georgios Alexandrakis, Arlington, TX (US); Bilal Khan, Fort Worth, TX (US)

(21) Appl. No.: 13/410,187

(22) Filed: Mar. 1, 2012

Related U.S. Application Data
(60) Provisional application No. 61/464,305, filed on Mar. 2, 2011.

(51) Int. Cl. A61B 6/00

(52) U.S. Cl. 600/473

(57) ABSTRACT

Disclosed is a functional NIRS imaging system including an elastomeric cap, a set of transmit optical fibers and a set of receive optical fibers terminating on the inside surface of the elastomeric cap. A pair of light sources combines to produce a collimated light beam at two wavelengths. An optical modulation system, converts the light beam into a plurality of probe light beams, modulates the plurality of probe light beams with a set of pseudo-orthogonal codes and directs each probe light beam into a transmit fiber. An optical detection system accepts scattered photons from subcutaneous tissue underneath the elastomeric cap as a plurality of collected light beams and converts them into a time series of electronic images, stores the electronic images into the memory and processes the electronic images using the pseudo-orthogonal codes. The system displays the resulting image on a display as a hemoglobin oxygen saturation map.

### CROSS-REFERENCE TO RELATED APPLICATIONS

This application claims priority to provisional application No. 61/464,305 filed on Mar. 2, 2011.

### FIELD OF THE INVENTION

The present disclosure relates to measurement and monitoring of near infrared images of subcutaneous matter. The field of the invention includes functional near infrared spectroscopic imaging of the brain and muscle tissues and includes dynamic imaging of hemoglobin oxygen concentration.

### BACKGROUND OF THE INVENTION

Non-invasive methods of monitoring brain activity have long been sought. More recently, systems that allow a patient to perform activities while monitoring brain activity, known as functional monitoring have been sought which allow neurological and psychological study of the patient. Many types of diagnoses and studies are made possible including studies that involve brain development in children, brain activity in mentally deficient patients, patients that have experienced brain damage or concussions, conditions such as migraines, and in the elderly, conditions such as Alzheimer's disease.

The technique of near infrared spectroscopy and functional near infrared spectroscopy have been shown to be useful in obtaining images of subcutaneous matter including fluids and solid matter. Near infrared spectroscopy is accomplished by transmitting light through the skin from a near infrared light source and into the subcutaneous tissue where it is scattered and absorbed. The scattered light is detected by a detector, usually coupled to an optical fiber placed a short distance away from the transmission source. Near infrared images of subcutaneous matter are generally wavelength dependent due to the light absorption characteristics of water, of oxygenated hemoglobin (HbO) and of deoxygenated hemoglobin (Hb). Observation of three dimensional spatial variations of hemoglobin concentration is possible using multiple wavelengths of light. Most often, lasers or LED sources in the ranges of 690 nm to 830 nm are employed.

Near-infrared spectroscopy has shown to be a promising tool in breast imaging for early detection of breast cancer, peripheral vasculature for diagnostics relating to peripheral blood flow and arterial health which are common issues in diabetics, and in related research applied to animals.

Depth of the imaging field is in the range of centimeters: about 3 to 4 cm for brain imaging, about 10-12 cm for breast imaging, and about 6 to 8 cm for the peripheral body such as the arms and legs.

Commercial systems exist in the art. FIG. 1A shows a typical prior art system. Patient 1 wears helmet 2. The helmet includes optical fibers held in close proximity to the patient's skull. The optical fibers include bundle of transmit fibers 3 and bundle of receive fibers 4. Bundle of transmit fibers 3 are connected to a set of lasers on a set of transmitter cards 5. Bundle of receive fibers 4 are connected to a corresponding set of optical detectors on a set of receiver cards 6. The set of transmitter cards and the set of receiver cards are controlled by computer 8 having memory 7. The computer operates to collect data from the receiver cards and process it using image processor 10 to determine various imaging data. For example, hemoglobin concentration may be determined as a function of position on the skull and at various depths within the brain. Display 9 is used to display the processed images and hemoglobin concentration maps.

In FIG. 1B, a prior art transmitter card is shown. The transmitter card includes a set of lasers 21 operating at the two wavelengths, 690 nm and 830 nm. Typically, there are four lasers of each wavelength on a card, where each laser includes fiber pigtail 25 to an external fiber optic connector 22. Fibers 27 from bundle of transmit fibers 3 are separated. Each fiber includes a connector which is mated to connector 22.

In FIG. 1C, a prior art receiver card is shown. The receiver card includes a set of detectors 18. Avalanche photodiode detectors (APD) are typically included for detecting low light levels. In most cases, four photodetectors are included on each card. Each photodetector includes fiber pigtail 20 to external fiber optic connector 23. Fibers 28 from the bundle of receive fibers are separated. Each fiber terminated with a fiber connector which is mated to external fiber optic connector 23. The transmitter cards and the receiver cards are connected to and interact with the computer.

FIG. 2 shows a prior art diagram depicting a detail of elastomeric cap 2 in operation. A patient's head including skin 39 covering subcutaneous fluid 40 is shown. Membrane 38 supports and positions input fiber 30, and output fibers 31-36. Near infrared light, when injected through input fiber 30 is scattered by the subcutaneous fluid in all directions. However, certain scattering paths allow for the light to propagate into the output fibers. An example is central blood flow artifact 48. Light path 44 captures light from input fiber 30 to output fiber 31. Light path 42 captures light from input fiber 30 to output fiber 32. Light path 43 captures light from input fiber 30 to output fiber 33. Light path 44 captures light from input fiber 30 to output fiber 34. Light path 45 captures light from input fiber 30 to output fiber 35. Light path 46 captures light from input fiber 30 to output fiber 36. The “banana” shaped light paths are statistical in nature describing paths that photons take while propagating in the subcutaneous fluid from the light source towards the detector. In so doing, some of the photons are absorbed. The absorption is exponentially related to the path length and to artifacts. In particular, photons propagating in the region of central blood flow artifact 48 will experience a higher rate of absorption as determined by the extinction coefficient of the material comprising central blood flow artifact 48. Thus, light detected in output fibers 33 and 35, in particular, will be less than their counterparts, output fibers 34 and 36, respectively. Also, note that output fibers 35 and 36 probe deeper into the subcutaneous fluid than output fibers 31 and 32, for example.

In the prior art, Grattan et al. in U.S. Pat. No. 5,497,769 discloses the quantitative determination of various materials in highly scattering media such as living tissue in an external, photometric manner by the use of a plurality of light sources positioned at differing distances from a sensor. The light from said sources is amplitude modulated in accordance with conventional frequency domain fluoremetry techniques where the gain of the sensor is modulated at a frequency different from the frequency of the light modulation. The sensor heads carry eight light sources and the light passing through the living tissue may be transmitted to a photomultiplier detector by an optical fiber.

Barbour et al., in U.S. Pat. No. 7,778,693 discloses a time series of optical tomography data obtained for a target tissue site in a human using a near infrared optical wavelength to observe properties of the vasculature of the human. A target placed in an imaging head is exposed to optical energy from combined sources. A source demultiplexer is controlled by a computer to direct the optical energy source fibers sequentially. The imaging head contains a plurality of source fibers and detector fibers for transmitting and receiving light energy, respectively. The optical energy entering the target at one location is scattered and may emerge at any location around the target where it is collected by detector fibers. The imaging process is repeated so as to deliver optical energy sequentially, a measurement being obtained for detected emerging optical energy at each detector for each emitting source fiber. Barbour et al. discloses a system with 32 detection channels.

In U.S. Pat. No. 7,983,740 to Culver et al., an imaging system for diffuse optical tomography is disclosed including a dense grid of light emitting diodes as sources wherein each light emitting diode has individual, isolated power to reduce crosstalk and each detector channel has a dedicated avalanche photo diode. The separation of signals is carried out through decoding frequency encoding.

These prior art systems suffer from a number of deficiencies. A first deficiency is in the size and portability of the system. As researchers and physicians have gained experience with these systems, they have seen the need for a larger numbers of detectors and sources in order to increase resolution. Current systems have as many as 128 detectors or transmitters. Such a system would require a fairly large rack of equipment and substantial space to operate. Furthermore, the sheer numbers and cost of the electronics become prohibitively large. Second, prior art systems operate at frame rates of about 2.5 Hz or less, at low resolutions and small coverage areas. For larger coverage areas the frame rates deteriorate to less than 1 Hz.

A compact near infrared hyperspectral imaging system is disclosed by Livingston et al. in U.S. Patent Application Publication No. 2008/0306337 that discloses an apparatus and method of the use of a hyperspectral surgical laproscope comprising a liquid crystal tunable filter mounted on the laproscope, positioned to collect back-reflected light from a target, and focal plane array also mounted on the laproscope to image light reflected from the target.

A digital light processing hyperspectral imaging apparatus is disclosed by Zuzak et al. in U.S. Patent Application Publication No. 2010/0056928, the system including an illumination source adapted to output a light beam, the light beam illuminating a target, a dispersing element arranged in the optical path and adapted to separate the light beam into a plurality of wavelengths, a digital micromirror array adapated to tune the plurality of wavelengths into a spectrum, an optical device having a detector and adapted to collect the spectrum reflected from the target and arranged in the optical path and a processor operatively connected to and adapted to control at least one of these components and further adapted to output a hyperspectral image of the target.

However, these prior art systems only provide images of the spectral response of the tissue area and depth illuminated as a whole without regard for localized photonic excitation and scattering. As a result, lateral and depth spatial resolutions as well as image contrast of these systems remain limited.

### SUMMARY OF THE INVENTION

A functional NIRS imaging system for hemodynamic imaging of subcutaneous tissue is disclosed utilizing a computer with a processor, a memory, a persistent storage device and a display. The system includes an elastomeric cap; a first optical fiber bundle terminating together at an entrance plane and terminating dispersively on the inside surface of the elastomeric cap; a second optical fiber bundle terminating together at an exit plane and terminating dispersively on the inside surface of the elastomeric cap. The system has a first light source producing a first light beam at a first wavelength and a second light source producing a second light beam at a second wavelength, both controlled by the computer. An optical combiner combines the first light beam and the second light beam into a third light beam. An optical collimator collimates the third light beam into a fourth light beam. An optical modulation system, controlled by a modulation controller, accepts the fourth light beam, converts the fourth light beam into a plurality of probe light beams, modulates the plurality of probe light beams and programmatically directs each probe light beam in the plurality of probe light beams into a corresponding optical fiber in the optical fiber bundle.

An optical detection system controlled by the first processor is connected to the first memory, which accepts a plurality of collected light beams from the second optical fiber bundle, converts the plurality of collected light beams into a time series of electronic images and stores the time series of electronic images into the memory.

The computer converts the stored time series of electronic images into a set of hemoglobin oxygen saturation level images, programmatically displays the set of hemoglobin oxygen saturation level images on the display and stores the set of hemoglobin oxygen saturation level images in the persistent storage device.

The first light source and the second light source may be controlled to alternate the wavelength of the fourth light beam between the first wavelength and the second wavelength.

The functional NIRS system further comprises a set of pseudo-orthogonal codes stored in the modulation controller, each pseudo-orthogonal code corresponding to each optical fiber in the first optical fiber bundle, the set of pseudo-orthogonal codes having a pre-defined code chip length c. The plurality of light beams are modulated with the set of pseudo-orthogonal codes. In another aspect, each probe light beam is uniquely modulated.

A first set of c raw images, corresponding to the first wavelength, as collected from the optical detection system is stored into the memory. Similarly, a second set of c raw images, corresponding to the second wavelength, as collected from the optical detection system is stored into the memory.

Where there are f fibers in the first fiber bundle there are a first set of f intermediate images derived from the first set of c raw images and the set of pseudo-orthogonal codes and a second set off intermediate images derived from the second set of c raw images and the set of pseudo-orthogonal codes.

A first resultant image, stored in the first memory, is derived from the first set off intermediate images and a physical transformation matrix. A second resultant image, stored in the first memory, is derived from the second set off intermediate images and the physical transformation matrix. From the two resultant images, a hemoglobin saturation map is derived and stored in the persistent storage device.

In another aspect of the invention, the modulation controller modulates the plurality of probe light beams according to a modulation scheme selected from the group consisting of time division multiple access modulation, frequency division multiple access modulation and code division multiple access modulation.

In one embodiment, the optical modulation system includes a spatial light modulator and an optical lens and in a first aspect of this embodiment the spatial light modulator is a MEMS digital mirror device. In a second aspect of this embodiment the spatial light modulator is a liquid crystal light modulator.

The optical detection system may include an optical lens and an optical detector array. In a first aspect of the optical detection system, the optical detector array includes an array of avalanche photodiodes. In an alternate embodiment, an array of regular photodiodes is employed. In a second aspect of the optical detection system, the optical detector array is a multi-anode photomultiplier tube. In a third aspect of the optical detection system, the optical detector array a CMOS imaging device. In a fourth aspect of the optical detection system, the optical detector array is a charge-coupled imaging device.

In another embodiment, the second optical fiber bundle terminates on the inside surface of the elastomeric cap in a packed hexagonal pattern with each receive optical fiber in the second optical fiber bundle located at a vertex of a hexagon. In one aspect of this embodiment each optical fiber in the first optical fiber bundle terminates on the inside surface of the elastomeric cap near the center of each hexagon in the packed hexagonal pattern. In another aspect of this embodiment, each hexagon in the packed hexagonal pattern has a characteristic size of about 2 millimeters. In another aspect of this embodiment, the first optical fiber bundle comprises about 4000 optical fibers and the second optical fiber bundle comprises about 8500 optical fibers and the elastomeric cap is approximately the size of a human scalp.

The functional NIRS system comprises a set of calibration data stored in the memory including an index of positions for the plurality of probe light beams, the position of each optical fiber in the first optical fiber bundle at the entrance plane, the position of each optical fiber in the second optical fiber bundle at the exit plane, an index of the terminated position of each optical fiber in the first optical fiber bundle as terminated on the elastomeric cap, an index of the terminated position of each optical fiber in the second optical fiber bundle as terminated on the elastomeric cap and an index relating each optical fiber in the second optical fiber bundle to a detector position in the optical detection system.

The functional NIRS system further comprises a data table stored in the first memory including an indexed assignment of each pseudo-orthogonal code in the set of pseudo-orthogonal codes to each optical fiber in the first optical fiber bundle, and the lateral positions of the each optical fiber as terminated on the elastomeric cap.

In an embodiment of the modulation scheme, the indexed assignment of the set of pseudo-orthogonal codes includes strict levels of orthogonality between optical fibers in the first optical fiber bundle that are terminated in close proximity at the elastomeric cap and further includes reduced levels of orthogonality between a second set of optical fibers and a third set of optical fibers, in the first optical fiber bundle, wherein the second set of optical fibers terminates near the center of the elastomeric cap and the third set of optical fibers terminates near the edge of the elastomeric cap.

A second embodiment of the functional NIRS imaging system for hemodynamic imaging of subcutaneous tissue includes a computer with a processor, a memory, a persistent storage device and a display. The system further comprises an elastomeric cap, an optical fiber bundle, terminating together at a first end plane and terminating dispersively on the inside surface of the elastomeric cap, comprising a set of transmit optical fibers and a set of receive optical fibers, a first light source producing a first light beam at a first wavelength and controlled by the computer, a second light source producing a second light beam at a second wavelength and controlled by the computer, an optical combiner combining the first light beam and the second light beam into a third light beam, an optical collimator collimating the third light beam into a fourth light beam, a modulation controller including a second processor and a second memory, and an optical modulation system, controlled by the modulation controller, which accepts the fourth light beam, converts the fourth light beam into a plurality of probe light beams, independently modulates each probe light beam in the plurality of probe light beams, programmatically directs each probe light beam in the plurality of probe light beams into a transmit optical fiber in the set of transmit optical fibers.

The functional NIRS imaging system programmatically directs a plurality of collected light beams from the set of receive optical fibers into an optical detection system, which is controlled by the first processor and connected to the first memory, and which accepts the plurality of collected light beams from the optical modulation system, converts the plurality of collected light beams into a time series of electronic images and stores the time series of electronic images into the memory.

A computer readable media including a first set of programmed instructions, when executed by the processor, converts the stored time series of electronic images into a set of hemoglobin oxygen saturation level maps, programmatically displays the set of hemoglobin oxygen saturation level maps on the display and stores the set of hemoglobin oxygen saturation level maps in the persistent storage device.

Also disclosed herein is a method for hemodynamic imaging of subcutaneous tissue utilizing a computer, with a first processor and a first memory, an optical modulator connected to a programmable modulation controller with a second processor and a second memory, and a detector array connected to the computer, the method providing for at least one optical fiber bundle including a set of transmit fibers terminating at the surface of an elastomeric cap and a set of receive fibers terminating at the surface of an elastomeric cap. The method further providing a collimated light beam, alternating the wavelength of the collimated light beam between wavelengths L1 and L2, dividing the collimated light beam into a set of probe beams in the optical modulator and modulating the set of probe beams with the optical modulator using a set of modulation codes.

The method continues by transmitting the set of probe beams through the set of transmit fibers and through the elastomeric cap wherein each probe beam is transmitted primarily by a single optical fiber. Photons scattered from the subcutaneous tissue below the elastomeric cap into the set of receive fibers are collected and delivered to the detector array wherein each fiber in the at least one optical fiber bundle is imaged onto a subset of detectors in the detector array to form a set of image data.

The method continues by a group of processing steps including processing the set of image data to create two high resolution images of the subcutaneous tissue wherein the two high resolution images include a first high resolution image corresponding to the collimated light beam at the wavelength L1 and a second high resolution image corresponding to the collimated light beam at wavelength L2, and combining the two high resolution images together to create a hemoglobin oxygen saturation image of the subcutaneous tissue.

In a second embodiment, the method provides a transmit optical fiber bundle for the set of transmit fibers and a receive optical fiber bundle for the set of receive fibers.

In a third embodiment, the method includes mapping each receive fiber in the set of receive fibers to each image pixel in the detector array corresponding to the each receive fiber's position on the elastomeric cap, and delivering the photons from the set of receive fibers to the detector array with the optical modulator according to the mapping.

The method continues by determining a fiber map P which maps each transmit fiber in the set of transmit fibers to a probe beam in the set of probe beams, assigns the set of modulation codes M of code chip length c, wherein one modulation code is assigned for each transmit fiber in the set of transmit fibers. The fiber map P is combined with the set of modulation codes M into a set of modulation matrices S and stored with the fiber map P, the set of modulation codes M and the set of modulation matrices S in the programmable modulation controller.

The method includes configuring the collimated light beam with light at the wavelength L1, modulating the set of probe beams with the set of modulation matrices S(tn) during time intervals tn=n Δt, where n=1 to the chip code length c and Δt is a predefined dwell time, reconfiguring the collimated light beam with light at the wavelength L2, and repeating the step of modulating the set of probe beams with the modulation matrices S(tn).

The method further includes recording a set of raw images X1 in the detector array after step b and before step c during the time intervals t1 to tc, recording a set of raw images X2 in the detector array after step d during the time intervals t1 to tc, correlating the set of raw images X1 to the modulation codes M to form a first set of intermediate images R1, correlating the set of raw images X2 to the modulation codes M to form a second set of intermediate images R2, combining the first set of intermediate images into a cap oriented image for wavelength L1, combining the second set of intermediate images into a cap oriented image for wavelength L2, and combining the cap oriented image for wavelength L1 with the cap oriented image for the wavelength L2 to arrive at a hemoglobin oxygen saturation image of the subcutaneous tissue.

The method includes a map function which converting an input set of intermediate images to physical dimensions of the elastomeric cap and maps an image pixel to a position on the elastomeric cap.

The method provides for creating and applying a transformation matrix that incorporates a model of physical transformations for improving image resolution and for applying a set of instrument calibrations including optical fiber losses. The result of the transformation matrix is a pair of images Iₕ, one image for HbO and one image for Hb concentrations. A single hemoglobin oxygen saturation image of the subcutaneous tissue is determined from the Iₕ by forming a ratio of HbO pixel values and (HbO+Hb) pixel values.

In another aspect of the method, the step of creating the transformation matrix includes incorporating reconstruction techniques to de-noise the first and second sets of intermediate images. In yet another aspect of the transformation matrix, a reconstruction technique is selected from the group consisting of: applying lower-level regularization in a Moore-Penrose inverse transformation, applying deblurring techniques based on Bayesian priors, applying synthetic aperture analysis and applying any combination thereof.

In yet another aspect of the method, the step of applying the transformation matrix is repeated to create a set of image pairs Iₕ(d) for varying depths d and the step of determining hemoglobin oxygen saturation image is repeated to determine a three-dimensional hemoglobin oxygen saturation image.

In relation to the modulation codes, one aspect of the method provides a set of pseudo-orthogonal codes as the set of modulation codes, and assigns the set of pseudo-orthogonal codes to the set of transmit fibers based on the potential for optical crosstalk.

Another aspect of the method, in relation to the modulation codes, a set of pseudo-orthogonal codes is provided as the set of modulation codes wherein a pseudo-orthogonal code is assigned to each transmit fiber in the set of transmit fibers. The method requires strict orthogonality between pairs of pseudo-orthogonal codes in the set of pseudo-orthogonal codes assigned to pairs of transmit fibers in the set of transmit fibers, wherein the pairs of transmit fibers terminate in close proximity to one another on the elastomeric cap. The method allows for reducing the code chip length of the set of pseudo-orthogonal codes by reducing the level of orthogonality between a first set of transmit fibers in the at least one optical fiber bundle, terminating near the center of the elastomeric cap and a second set of transmit fibers in the at least one optical fiber bundle, terminating near the edge of the elastomeric cap.

In another aspect of the method, in relation to the modulation codes, the method provides a set of pseudo-orthogonal codes as the set of modulation codes wherein a pseudo-orthogonal code is assigned to each transmit fiber in the set of transmit fibers. Furthermore, the same level of orthogonality is assigned to each pseudo-orthogonal code in the set of pseudo-orthogonal codes based on a set of cross-correlation coefficients.

A more detailed discussion of these embodiments and aspects of the embodiments are provided herein.

### BRIEF DESCRIPTION OF THE DRAWINGS

FIGS. 1A, 1B and 1C depict systems of the prior art.

FIG. 2 is a diagram showing the propagation and scattering of light from a transmit fiber to a set of receive fibers.

FIG. 3 is a block diagram of an fNIRS system according to a first embodiment.

FIG. 4 is diagram of a section of an elastomeric cap of a preferred embodiment.

FIG. 5 is a calibration table related to the optical transmission.

FIG. 6 is a calibration table related to the optical detection.

FIG. 7 is a flow chart for the overall operation of an fNIRS system.

FIG. 8A is a flow chart of a calibration method.

FIG. 8B is a flow chart of an optical transmission method used during operation of an fNIRS system.

FIG. 9 is block diagram depicting the mathematical operations related to the optical transmission method.

FIG. 10 is a flow chart of an optical detection and processing method used during operation of an fNIRS system.

FIG. 11 is a block diagram of an fNIRS according to an alternate embodiment.

FIGS. 12A and 12B are diagrams of a fiber tip according to an alternate embodiment.

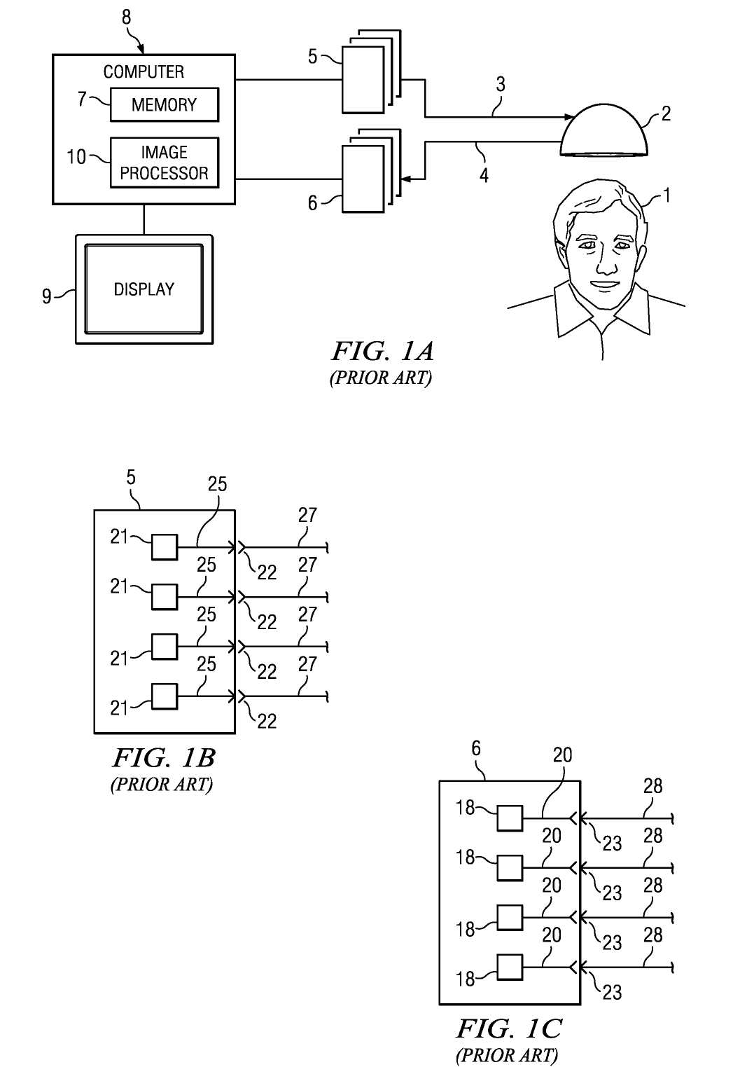

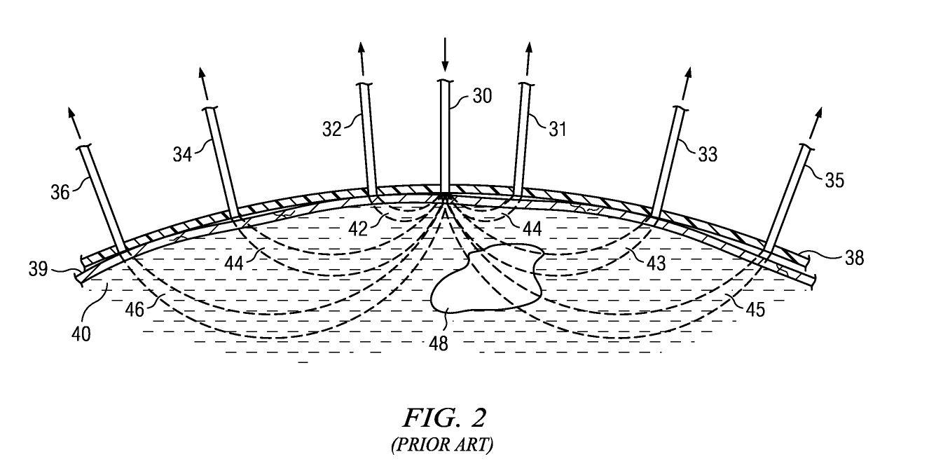

### DETAILED DESCRIPTION

Disclosed are a system and method for functional near infrared spectroscopy (fNIRS) that is useful for hemodynamics and provides a significant improvement over the prior art in resolution and frame rate of images.

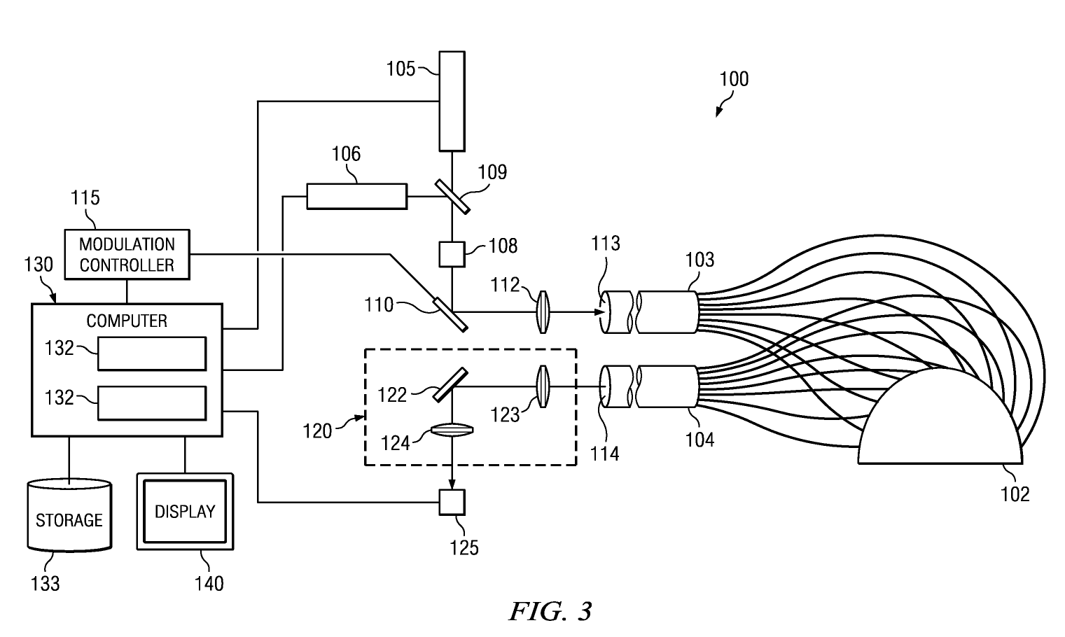

Referring to FIG. 3, an fNIRS system 100 is shown. Light source 105 operates at a first wave length λ1 in the range of 690 nm and is positioned to impinge an incident beam on combiner 109. Light source 106 operates at a second wave length λ2 in the range of 830 nm and is positioned to impinge an incident beam on combiner 109. The wavelengths are chosen to be on different sides of the equi-absorption wavelength for oxygenated hemoglobin and deoxygenated hemoglobin that occurs near 800 nm. Combiner 109 combines the light beam from light source 105 with the light beam from a light source 106 into a transmit beam which is directed toward collimator 108. Collimator 108 expands and collimates the transmit beam and directs it toward modulator 110. Modulator 110 is a spatial light modulator such as a MEMS device. In the preferred embodiment, the MEMS device is a digital mirror device (DMD) available from Texas Instruments Corporation. The DMD device is a controllable two-dimensional ray of MEMS mirrors. Other types of special light modulators can be employed. Modulator 110 is logically connected to modulation controller 115 and responds to commands received from it.

Modulator 110 redirects the incident beam to lens 112. Lens 112 focuses the transmit beam onto transmit fiber bundle 103 at input termination plane 113. Transmit fiber bundle 103 in the preferred embodiment is a series of plastic fibers of diameter 0.5 mm to 1.0 mm. In the preferred embodiment, the number of transmit fibers in transmit fiber bundle 103 can range between 5,000 and 50,000 individual fibers.

Transmit fiber bundle 103 terminates in a distribution of individual fibers into elastomeric cap 102. Each of the fibers is fixed in the elastomeric cap and directs portions of the transmit beam toward the patient.

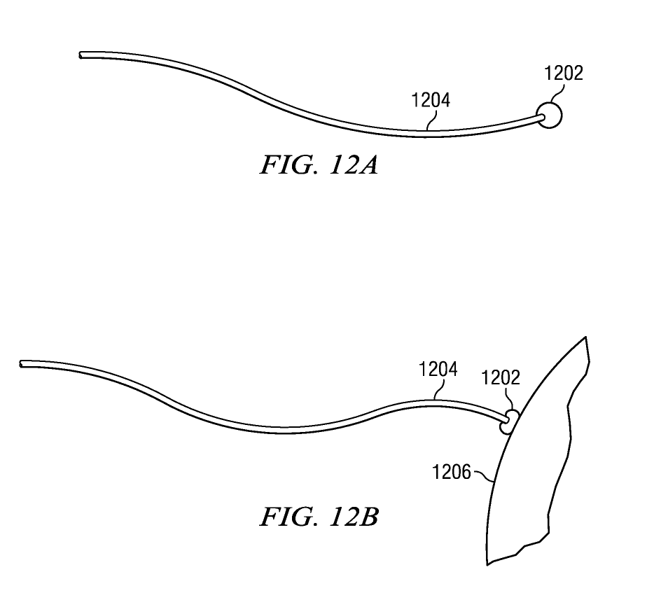

A drawback of prior art implementation is that the hard tip of the fiber on the patient's scalp may cause discomfort. As shown in FIGS. 12A and 12B, a general solution to this is to cushion the contact of each fiber against the scalp. An example of this is to adhere a soft polymer (e.g. PDMS) bead 1202 onto the tip of each fiber 1204. The size of this bead may be optimized to still thread through a particular hair type and still cushion contact with the scalp 1206. This material may be pliable and optically transparent. The patient or subject experience is improved. In addition the coupling coefficient between the fiber and the scalp is improved, in some cases by 2 dB due to the soft polymer tip over a fiber without the tip. As would be recognized by one skilled in the art, other methods of cushioning the tip, for example, a spring-like holder for the fibers, will also improve the patient's comfort.

Returning again to FIG. 3, scattered beams are received at individual fibers of receive fiber bundle 104 at elastomeric cap 102. Receive fiber bundle 104 terminates at output termination plane 114. Lens 123, lens 124 and reflector 122 cooperate to arrange and magnify the image received at termination plane 114 and focus it to detector array 125.

Each portion of the multiple portions of the transmit beam is preferably associated to a mirror device or cluster of mirrored devices in modulator 110. Lens 112 in combination with the adjustable mirror positions focuses the portion of light reflected from each mirror device to a single fiber at the input termination plane 113. To modulate the light propagating into a single fiber, the associated mirror device position is modulated according to a set of modulation codes. The modulation for a single mirror device and associated fiber is preferably done to either focus all of the portion of light onto the single fiber or deflect the portion of light away from the system optics all together. The transmit-receive channels are isolated from other nearby transmit-receive pairs by the use of orthogonal modulation at the transmitter and concomitant demodulation at the receiver. Examples of orthogonal modulation are well known in the art and include Time Division Multiple Access (TDMA), Frequency Division Multiple Access (FDMA) or Code Division Multiple Access (CDMA). Each of these or a combination of these is useful in the invention.

Modulation controller 115 is programmed to modulate the minor devices of modulator 110. In alternate embodiments, the modulation controller is programmed to modulate the optical elements to control light intensity of the multiple portions of the transmit beam, for example, to modulate cells of liquid crystal light modulator.

Detector array 125 is selected from the group of an array of avalanche photodiodes (APD), an array of silicon photodiodes, a cooled, high sensitivity solid state detector array, a multi-anode photomultiplier tube, a CMOS imaging device, and a charge coupled (CCD) imaging device. A multi-anode photomultiplier tube is preferably used for detector array 125.

Computer 130 includes electronic memory 131, such as random access memory, and persistent electronic storage 133, such as a hard drive. Computer programs are stored on persistent electronic storage 133 and loaded into electronic memory 131 to make computer 130 operable to perform the functions of the exemplary embodiments of the present invention. One set of such computer programs, image construction 132, operates to collect raw data from detector array 125, construct images relating to subcutaneous materials of the patient's brain from the raw data, and display the images and related results on display 140. In the preferred embodiment, the images of image construction 132 are image maps of hemoglobin oxygen saturation across the patient's brain at various depths within the patient's skull.

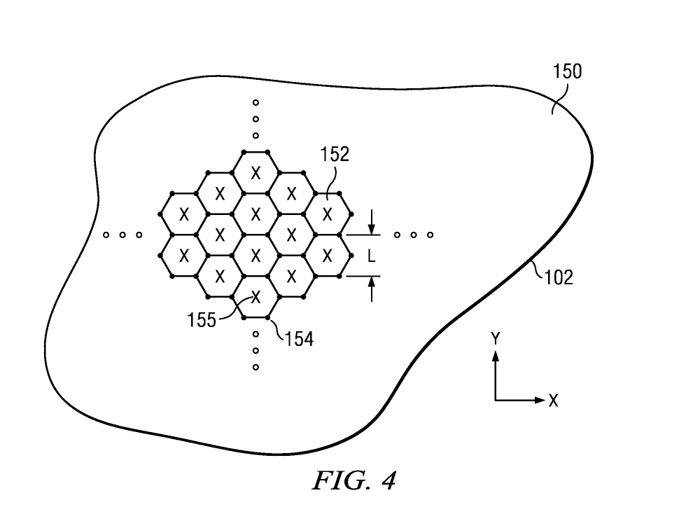

FIG. 4 is a diagram of a preferred embodiment elastomeric cap 102. Each dot in a set of dots 154 represents a receive fiber position for receive fibers in the receive fiber bundle. Each X in a set of X's 155 mark a transmit fiber position for transmit fibers in the transmit fiber bundle. The receive fiber positions are arranged in a set of hexagonal units 152 which are packed together to share receive fibers. The transmit fiber positions are placed at the center of each hexagonal unit in the set of hexagonal units. The set of hexagonal units, receive fibers and transmit fibers are spread over the entire surface 150 of the elastomeric cap 102.

In the preferred embodiment the characteristic size of a hexagonal unit is about 2 mm. Since the elastomeric cap covers approximately half of the patient's head, a typical elastomeric cap wilt contain about 4000 hexagonal units, 4000 transmit fibers and about 8500 receive fibers. Thus, a preferred transmit fiber bundle includes about 4000 optical fibers and a preferred receive fiber bundle includes about 8500 optical fibers. The ratio and numbers of transmit fibers and receive fibers can vary. The physical arrangement of the transmit and receive fibers can also vary. In general, it will be desirable to place fibers on 1 mm or less spacings in other to achieve mm or sub-millimeter resolution. In addition, thin, flexible, plastic fibers will easily thread through the hair of the subject under test.

The (NIRS system must be calibrated to include calibration data relating to (1) an indexing of positions of DMD mirror devices in relation to lateral positions of the portions of the transmit beam in the transmit termination plane, (2) the position of fibers in the receive termination plane as imaged on the image plane, (3) an indexing of transmit fiber positions as terminated on the elastomeric cap in relation to the lateral positions of each transmit fiber in the transmit termination plane, (4) an indexing of receive fiber positions as terminated on the elastomeric cap in relation to the lateral positions of each receive fiber in the receive termination plane, an indexing of each receive fiber upon a detector pixel or set of detector pixels on the receiving array, and (6) a set of pseudo-orthogonal codes chosen for the modulation codes, each pseudo-orthogonal code assigned to a transmit fiber in the transmit fiber bundle and indexed as in (3).

The set of pseudo-orthogonal codes are preferably chosen as known in the art of CDMA communications systems, typically consisting of modulating a signal with a code having “c” chips with a certain number “r” of occupied chips per code. The level of orthogonality is determined by computing cross-correlations between the codes. The number of transmit fibers relates to the level of orthogonality, the code length c and the number r. In the exemplary embodiments herein, the level of orthogonality of the codes can be chosen based on the potential for optical crosstalk so as to reduce the code length c. In a preferred embodiment a small code length is used to increase the frame rate. Also, transmit fibers near the edge of the elastomeric cap have very low probability of exciting receive fibers near the center of the elastomeric cap. It is advantageous to allow low levels of orthogonality between the transmit fibers far displaced, while enforcing strict orthogonality between transmit fibers in close proximity. In any alternate embodiment, all codes are chosen to have the same level of orthogonality based on a set of cross-correlation coefficients.

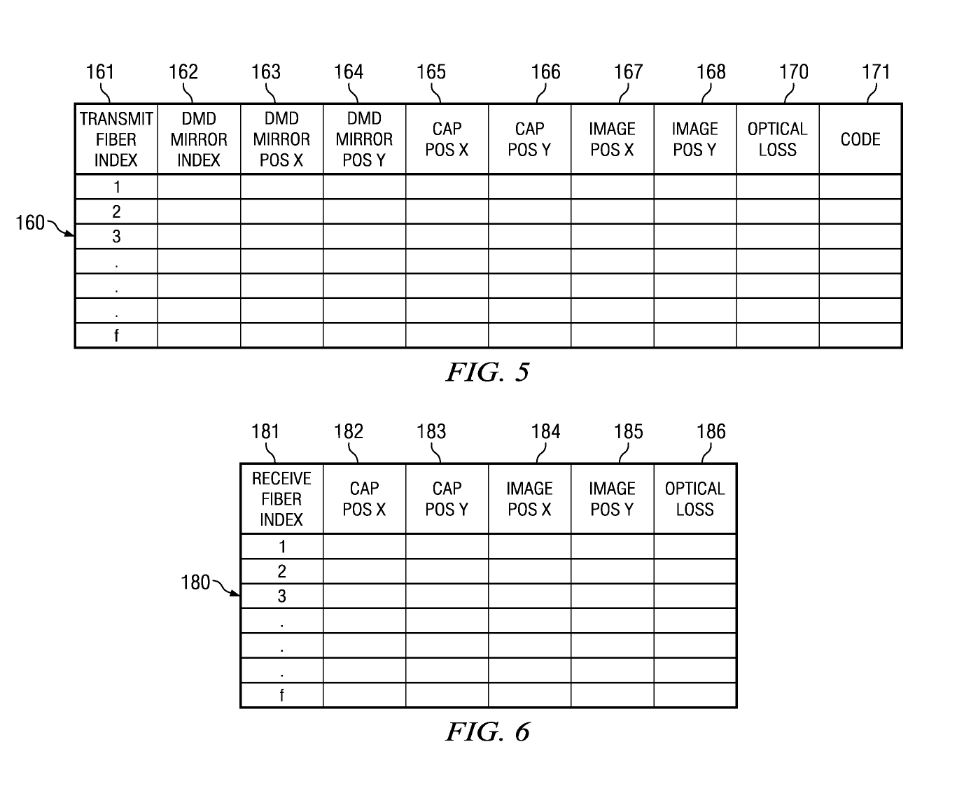

FIG. 5 is a calibration table describing the calibration aspects of the optical transmission components. Calibration table 160 includes one row for each transmit fiber in the transmit fiber bundle. Each row has a set of columns of associated calibration data. Column 161 provides an index to each transmit fiber. Column 162 provides a DMD mirror association. The association to the DMD mirror device in a preferred embodiment is a pair of indices, one index for each dimension of the device, as in a matrix position. Column 163 provides a DMD mirror x-position. The DMD mirror x-position correlates to a voltage applied to the DMD mirror device in one dimension to connect a light path to the associated transmit fiber. Column 164 provides a DMD mirror y-position. Similarly, the DMD mirror y-position is a voltage to be applied to the DMD mirror device in the second dimension to connect a light path to the associated transmit fiber. Column 165 is an x-position of the associated transmit fiber on the elastomeric cap. Column 166 is a y-position of the associated transmit fiber on the elastomeric cap. Column 167 is a pixel x-position of the associated transmit fiber within a reconstructed image of the elastomeric cap. Column 168 is a pixel y-position of the associated transmit fiber within a reconstructed image of the elastomeric cap. Column 170 is the optical loss measured through the system for the associated transmit fiber. Column 171 contains the modulation code for the associated transmit fiber, which is chosen from one of a hexadecimal and decimal representation of the binary equivalent of the modulation code, where each chip is a bit containing “1” or “0”. Alternatively, a set of columns is included in calibration table 160 each column containing a chip value.

FIG. 6 is a calibration table describing the calibration aspects of the optical detection components. Calibration table 180 includes one row for each receive fiber in the receive fiber bundle. Each row has a set of columns of associated calibration data. Column 181 provides an index to each receive fiber. Column 182 is a x-position of the associated receive fiber on the elastomeric cap. Column 183 is a y-position of the associated receive fiber on the elastomeric cap. Column 184 is a pixel x-position for the associated receive fiber within a reconstructed image of the elastomeric cap. Column 185 is a pixel y-position for the associated receive fiber within a reconstructed image of the elastomeric cap. Column 186 is the optical loss measured through the system for the associated receive fiber.

Calibration of the elastomeric cap is performed by imaging the inner surface of the elastomeric cap, causing light from a light source to propagate down individual fibers, and indexing the position of the fiber from which light emanates.

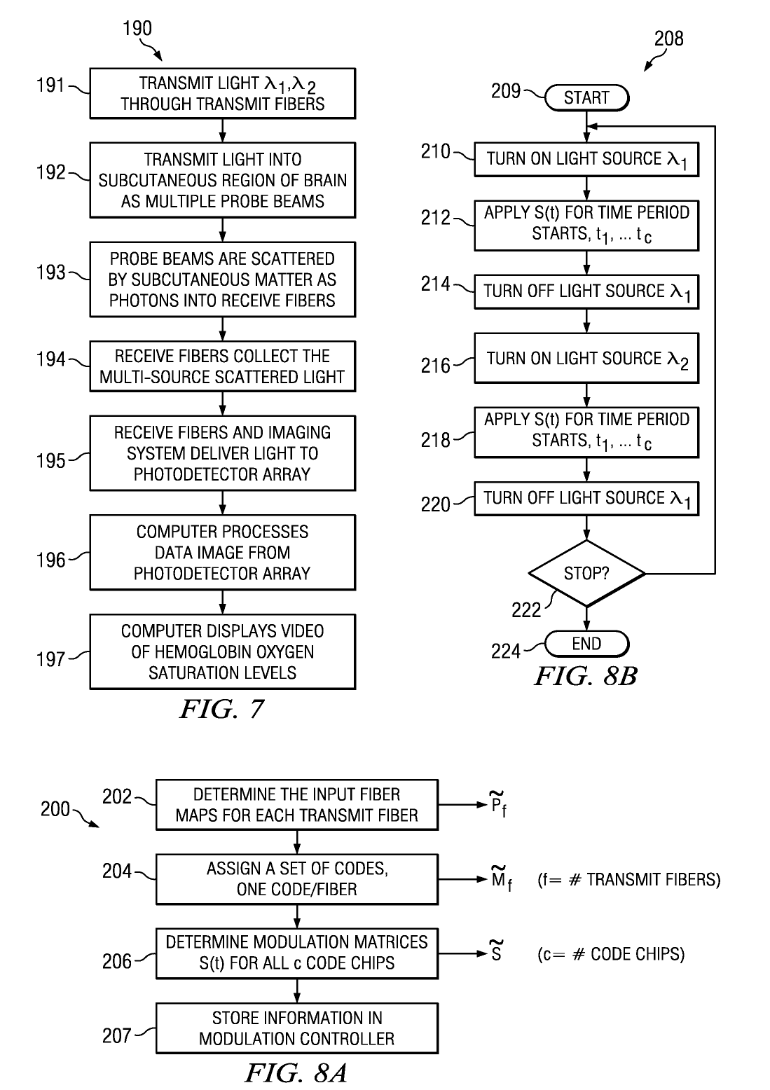

FIG. 7 shows an operational flow diagram 190 of the apparatus in operation. At step 191, the fNIRS system alternatively transmits collimated light of wavelengths λ1 and λ2 through a set of transmit fibers in transmit fiber bundle, and at step 192 the collimated light is split into a plurality of probe light beams and is further transmitted through the elastomeric cap, and into the subcutaneous matter of the patient's head. At step 193, the plurality of probe light beams experience optical scattering and absorption from the subcutaneous matter to produce a multi-source scattered light. At step 194, scattered photons are collected by a set of receive fibers of the receive fiber bundle, wherein each receive fiber collects the multi-source scattered light. At step 195, the scattered photons when collected are delivered by each receive fiber and by the optical imaging system onto the detector array in predefined detector positions related to the order of receive fibers in the receive fiber bundle. At step 196, the detector array converts the scattered, collected and delivered photons into image data which the computer collects from the detector, reorders according to physical receive fiber position on the elastomeric cap and further processes the image data to form a time series of image frames of hemoglobin oxygen saturation level maps. At step 197, the time series of image frames of hemoglobin oxygen saturation levels are displayed as a real-time video on a display. These general steps are described in greater detail in the Figures that follow.

Referring to FIGS. 5 and 8A, calibration process 200 of the optical transmission is shown. At step 202, a set of positional maps are created which map the transmit fibers to a DMD mirror device as identified in a DMD modulation matrix. Step 202 creates and utilizes columns 161 and 162 of calibration table 160. At step 204, a set of modulation codes are assigned, one modulation code for each transmit fiber. Step 204 creates and utilizes columns 161 and 171 of calibration table 160. At step 206, a set of DMD modulation matrices are created based on the positional maps and the set of modulation codes. In step 206, each DMD modulation matrix comprises a master chip code to modulate the light intensities of all transmit fibers in the transmit fiber bundle. At step 207, the information generated in steps 202, 204 and 206 are stored in the modulation controller.

Referring to FIGS. 3 and 8B, operational process 208 of the system is shown. At step 209, the fNIRS system is made to start as a part of an fNIRS imaging run. At step 210 light source 105 is turned on to create a transmit beam. At step 212, the DMD modulation matrices are applied by modulation controller 115 to the modulator 110 to modulate the incident beam of λ1 by the assigned modulation code. Each ray from the modulator is transmitted into a specific transmit fiber of transmit fiber bundle 103. Step 212 is repeated until all codes have been transmitted. At step 214, light source 105 is turned off. At step 216, the light source 106 is turned on. At step 218, the DMD modulation matrices are applied by the modulation controller to modulator 110 to modulate the incident beam λ2 by the assigned modulation code. Each ray of light is transmitted into a transmit fiber of the transmit fiber bundle. Step 218 is repeated until all codes have been sent. At step 220, light source 106 is turned off. At step 222, the system checks if an operator has requested that the imaging run stop. If stopped, then method 200 ends at step 224, otherwise the method refers to step 210.

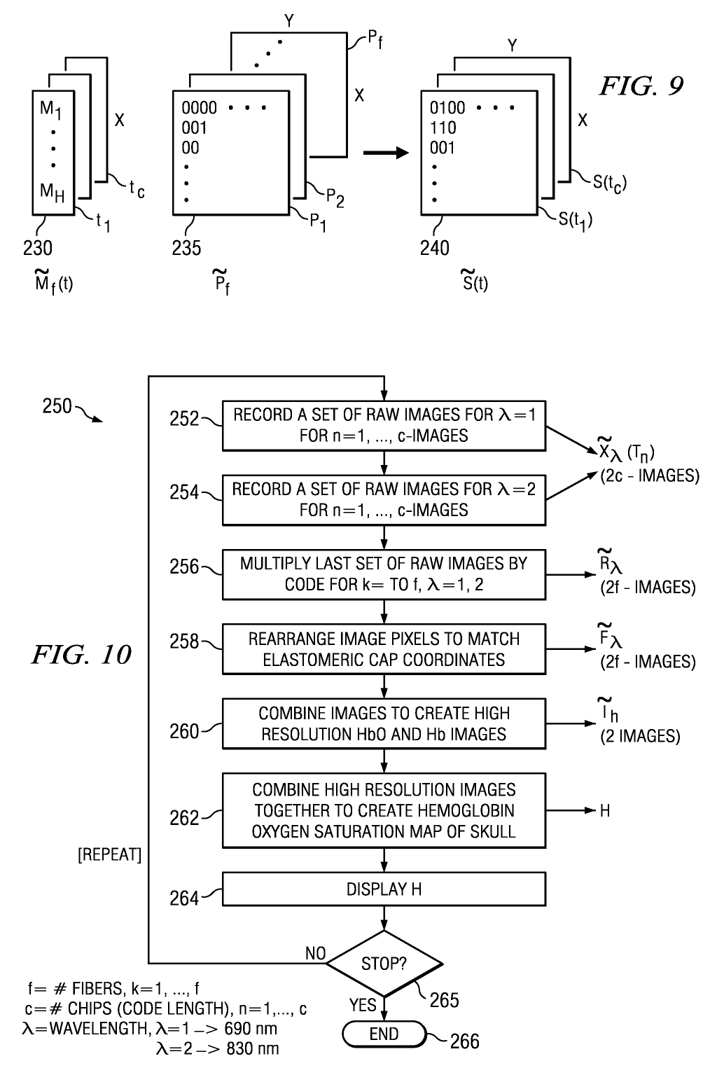

FIG. 9 describes the steps 202, 204 and 206 in greater detail. There are f transmit fibers in the transmit fiber bundle and c chips in each modulation code. The set of modulation codes M_{f}(t) 230 are arranged as vectors of chips (code bits), one vector for each chip with f elements indexed to each transmit fiber. The Mₖ(tₙ) is a code bit associated to transmit fiber k for code chip n to be applied at time tₙ, where tₙ=n Δt and Δt is a dwell time. The set of fiber positions are described by a set of positional matrices P_{f} 235, one positional matrix Pₖ for each transmit fiber k, where the x and y DMD matrix assignment is a “1” in the associated transmit fiber position. The set of modulation codes M_{f}(t) 230 and the set of positional matrices P_{f} 230 are combined to form a set of DMD modulation matrices S(t) 240 according to:

$$S ( {t}_{n} ) = {\sum }_{i = 1}^{f} {P}_{i} \cdot {M}_{i} ( {t}_{n} ) \qquad ( 1 )$$

where S(tₙ) is the DMD modulation matrix for modulating the DMD minor devices a time t associated to chip n where “1” in each matrix element means to apply light from the transmit beam to the transmit fiber associated to that DMD mirror device and where “0” in each matrix element means to apply no light to the transmit fiber associated to that DMD mirror device.

FIG. 10 is flow chart of optical detection and processing method 250. The assumptions surrounding the steps of optical detection and processing method 250 are that there are f fibers in the transmit fiber bundle, numbered k=1, . . . f; there are c chips in the modulation code (code length=c), numbered n=1, . . . c; there are two transmit beam wavelengths λ where λ=1 signifies a first probe wavelength, preferably chosen to be 690 nm, and λ=2 signifies a second probe wavelength, preferably chosen to be 830 nm. At step 252, a set of raw images X₁(tₙ) for a the first probe wavelength is recorded by the photodetector array and transferred to the computer memory where it is stored. At step 254, a set of raw images X₂(tₙ) for a the second probe wavelength is recorded by the photodetector array and transferred to the computer memory where it is stored. The result of steps 252 and 254 are the set of 2c images X_{λ}(tₙ) each image having the dimensions of the photodetector array.

At step 256 each set of raw images are correlated to the set of modulation codes according to:

$${R}_{λ kij} = {\sum }_{n = 1}^{c} {X}_{λ ij} ( {t}_{n} ) \cdot {M}_{k} ( {t}_{n} ) \qquad ( 2 )$$

where the set of intermediate images R_{λk} is calculated for both wavelengths k, for k=1 to f transmit fibers, and where Mₖ(tₙ) is the modulation code, preferably a pseudo-orthogonal code, associated to the kth transmit fiber. The indexes i and j range over the pixels of the sets of raw images. There are 2f intermediate images in the set of intermediate images, each intermediate image having dimensions of the photodetector array and each intermediate image related specifically to the response of the subcutaneous matter to a single fiber.

At step 258, the set of intermediate images are transformed to map the set of intermediate images into the coordinates of the elastomeric cap to create a set of cap oriented images F_{λk} according to:

F_{λk}=map(R_{λk}). (3)

Step 258 can employ a simple pixel rearrangement in some embodiments, while in other embodiments step 258 performs a scaling procedure to map pixels to real dimensions. Step 258 uses the information stored in columns 182, 183, 184 and 185 of table 180 to map an image pixel to an elastomeric cap position. The output of step 258 is the set of cap oriented images F_{λk} containing 2f images.

At step 260 the cap oriented images are further combined into two high resolution images, one image representing HbO concentration and one image representing Hb concentration according to the transformation:

Iₕ=AF_{λk} (4)

where the transformation matrix A incorporates the optical physics of combining multiple images at two wavelengths λ, normalizing the image according to the optical losses at each wavelength as contained in tables 160 and 180, and by determining extinction ratios of HbO and Hb, and calculating concentrations for h=HbO and Hb. It is possible to incorporate many physical and mathematical optimizations into the transformation matrix A. Some examples are: applying image re-construction techniques to de-noise the images such as the use of lower levels of regularization in a Moore-Penrose inverse, applying deblurring techniques based on Bayesian priors to create a super-resolution image, and applying synthetic aperture analysis to speed the computation and to allow for further increase in image resolution. The foregoing techniques can be applied in combination. The output of step 260 is two high resolution images Iₕ of the scattering return from subcutaneous matter in the patient's brain at the two probe wavelengths. In an alternate embodiment, multiple images Iₕ can be selectively created at differing depths for HbO and Hb concentration at the output of step 260 by applying different matrices A corresponding to gathering optical information at multiple depths within the patient's brain.

At step 262, the two high resolution images L are further combined to create a single hemoglobin oxygen saturation image, H, of the patient's brain by forming a ratio of the pixel values in the I_{HbO} image to the pixel values in the summed (I_{Hb}+I_{HbO}) image. The output of step 262 is a single two-dimensional image. In the alternate embodiment, step 262 is repeated for the multiple images to create hemoglobin oxygen saturation maps at different depths in the patient's brain. The output of the alternate embodiment is a three-dimensional image. At step 264, the two-dimensional hemoglobin saturation image is displayed on the display connected to the computer. In the alternate embodiment a three-dimensional image of hemoglobin saturation is displayed.

At step 265, the system checks if an operator has requested that the imaging run stop. If stopped, then the optical detection and processing method 250 ends at step 266, otherwise the optical detection and processing method 250 repeats at step 252.

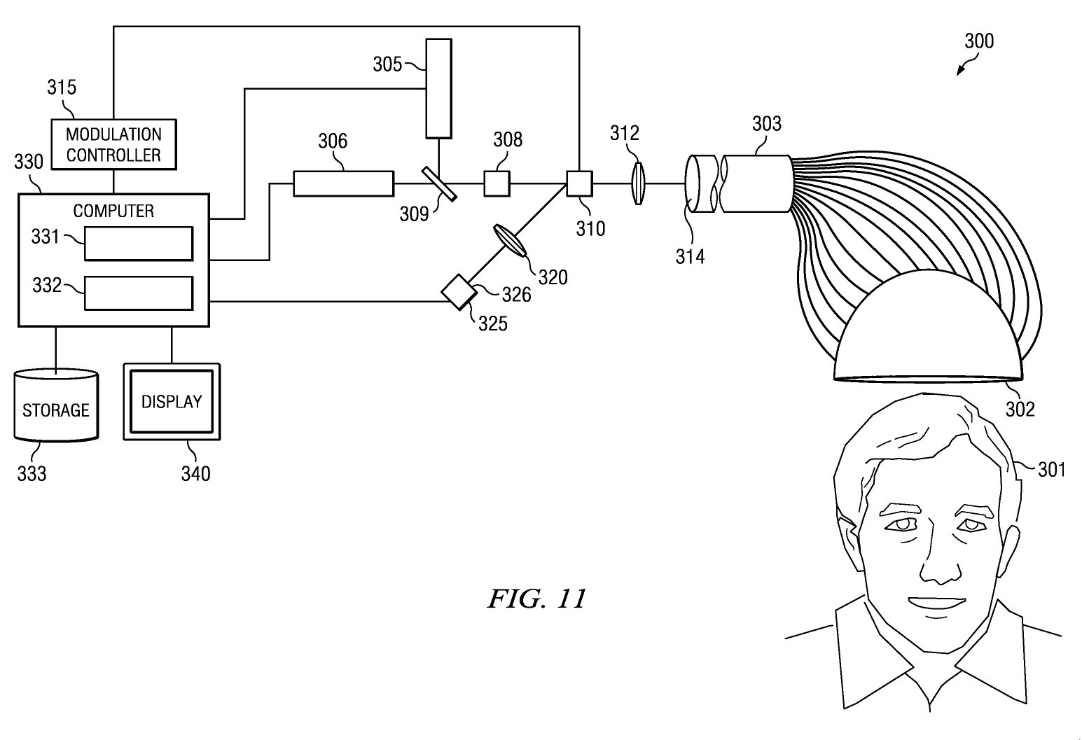

FIG. 11 shows a second embodiment of an fNIRS system 300. The system comprises two light sources: light source 305 operating at a first wavelength, λ1 and light source 306 operating at a second wavelength λ2, beam combiner 309 for combining a light beam from light source 305 with a light beam from light source 306 into a transmit beam, collimator 308 for expanding and collimating the transmit beam, bidirectional fiber bundle 303, spatial light modulator 310 for modulating multiple portions of the transmit beam, lens system 312 for focusing the multiple portions of the transmit beam onto multiple fibers of bidirectional fiber bundle 303 at optical termination plane 314, elastomeric cap 302 on which bidirectional fiber bundle 303 is fastened and optically terminated near the skull of patient 301, photodetector array 325, lens system 320 for imaging the optical termination plane 314 of bidirectional fiber bundle 303 onto photodetector array 325, computer 330 for processing images, a display 340 for displaying images, and modulation controller 315 for controlling spatial light modulator 310.

The wavelength λ1 is preferably 690 nm and wavelength λ2 is preferably 830 nm. The light sources are preferably lasers, and the spatial light modulator is preferably selected from group of a MEMS device and a liquid crystal light modulator. The optical fibers in bidirectional fiber bundle 303 are preferably low loss (per-fluorinated) plastic fibers of diameter 0.5 mm to 1.0 mm.

Each portion of the multiple portions of the transmit beam is preferably associated with a modulated element of spatial light modulator 310. Lens 312 in combination with spatial light modulator 310 focuses the light ray from each modulated element to a single fiber at the optical termination plane 314.

Imaging system 320 is preferably a lens system, in combination with spatial light modulator 310, arranged to create a magnified image of the optical termination plane 314 of the bidirectional fiber bundle on image plane 326 of photodetector array 325.

Modulation controller 315 is programmed to modulate the spatial light modulator so as to transmit light applied to the bidirectional fiber bundle and for receiving light collected by the bidirectional fiber bundle, redirecting the receive light into the imaging system 320.

Computer 330 includes processors, field programmable gate arrays, electronic memory 331, such as random access memory, and persistent electronic storage 333, such as a hard drive. Computer programs are stored on persistent electronic storage 333 and loaded into electronic memory 331 to make computer 330 operable to perform the functions of the exemplary embodiments of the present invention. One set of such computer programs, image construction 332, operates to collect raw data from photodetector array 325, construct images relating to subcutaneous materials of the patient's brain from the raw data, and display the images and related results on display 340. In the preferred embodiment, the images of image construction 332 are image maps of hemoglobin oxygen saturation across the patient's brain at various depths within the patient's skull.

Referring to FIG. 3, in a third embodiment of the present invention, a spatial light modulator is included in the optical imaging system 120. The spatial light modulator is programmed to redirect the light from each fiber in the receive fiber bundle to an image pixel which closely corresponds to a correct position of the receive fiber as it is terminated on the elastomeric cap. The third embodiment is configured to optically accomplish the step 258 of optical detection in processing method 250.

Other embodiments are envisioned. As for the apparatus, light sources are selected from the group of lasers, LEDs, broad area devices all of which operate at typical NIRS wavelengths. Modulation of the transmit beam can be effected at the light sources for certain aspects, such as synchronization. In other aspects, wherein there is an array of light sources, the array of light sources can be individually modulated instead of using the spatial light modulator. Modulation schemes can be selected from the group of CDMA, TDMA, FDMA in which code, timeslot and frequency, respectively, are reused across the area of the elastomeric cap providing distances between transmit fibers are large enough to avoid crosstalk. As for detection, the detector bundle can be directly pigtailed to an array of photodetector devices. Other imaging devices such as high resolution cameras may also be employed. The optical imaging system can utilize lenses, lens array prisms, and other known optical components used in massively connected optical systems. Elastomeric cap materials include any shape memory materials including polymers that provide for automated threading of fibers into the scalp of the patient.

1. A functional NIRS imaging system for hemodynamic imaging of subcutaneous tissue comprising:
a computer further comprising a first processor, a memory, a persistent storage device and a display;
an elastomeric cap;
a first optical fiber bundle terminating together at an entrance plane and terminating dispersively on the inside surface of the elastomeric cap;
a second optical fiber bundle terminating together at an exit plane and terminating dispersively on the inside surface of the elastomeric cap;
a first light source producing a first light beam at a first wavelength and controlled by the computer;
a second light source producing a second light beam at a second wavelength and controlled by the computer;
an optical combiner combining the first light beam and the second light beam into a third light beam;
an optical collimator collimating the third light beam into a fourth light beam;
a modulation controller including a second processor and a second memory;
an optical modulation system, controlled by the modulation controller, which accepts the fourth light beam, converts the fourth light beam into a plurality of probe light beams, modulates the plurality of probe light beams and programmatically directs each probe light beam in the plurality of probe light beams into a corresponding optical fiber in the first optical fiber bundle;
an optical detection system controlled by the first processor and connected to the first memory, which accepts a plurality of collected light beams from the second optical fiber bundle, converts the plurality of collected light beams into a time, series of electronic images and stores the time series of electronic images in the memory; and,
a computer readable media including a first set of program instructions, when executed by the first processor, converts the stored time series of electronic images into a set of hemoglobin oxygen saturation level maps.

2. The functional NIRS system of claim 1 wherein the first set of program instructions, that when executed by the first processor, controls the first light source and the second light source to alternate the wavelength of the fourth light beam between the first wavelength and the second wavelength.

3. The functional NIRS system of claim 1 further comprising a set of pseudo-orthogonal codes stored in the first memory and the second memory, each pseudo-orthogonal code corresponding to each optical fiber in the first optical fiber bundle, the set of pseudo-orthogonal codes having a pre-defined code length c.

4. The functional NIRS system of claim 3 wherein the computer readable media includes a second set of program instructions, that when executed by the second processor, modulates the plurality of probe light beams with the set of pseudo-orthogonal codes.

5. The functional NIRS system of claim 3 wherein each probe light beam in the plurality of probe light beams is modulated by a pseudo-orthogonal code in the set of pseudo-orthogonal codes.

6. The functional NIRS system of claim 5 comprising:
a first set of c raw images stored by the optical detection system in the first memory and corresponding to the fourth light beam having the first wavelength; and,
a second set of c raw images stored by the optical detection system in the first memory and corresponding to the fourth light beam having the second wavelength.

7. The functional NIRS system of claim 6 wherein there are f fibers in the first fiber bundle and further comprising
a first set off intermediate images derived from the first set of c raw images and the set of pseudo-orthogonal codes; and,
a second set off intermediate images derived from the second set of c raw images and the set of pseudo-orthogonal codes.

8. The functional NIRS system of claim 7 further comprising:
a first resultant image stored in the first memory and derived from the first set of f intermediate images and a transformation matrix; and,
a second resultant image stored in the first memory and derived from the second set off intermediate images and the transformation matrix.

9. The functional NIRS system of claim 8 comprising a hemoglobin saturation map stored in the persistent storage device and derived from the first resultant image and the second resultant image.

10. The functional NIRS system of claim 1 wherein the optical modulation system modulates light according to an orthogonal modulation selected from the group consisting of time division multiple access modulation, frequency division multiple access modulation and code division multiple access modulation.

11. The functional NIRS system of claim 1 wherein the optical modulation system includes a spatial light modulator and an optical lens.

12. The functional NIRS system of claim 11 wherein the spatial light modulator is a MEMS digital mirror device.

13. The functional NIRS system of claim 11 wherein the spatial light modulator is a liquid crystal light modulator.

14. The functional NIRS system of claim 1 wherein the optical detection system includes an optical lens and an optical detector array.

15. The functional NIRS system of claim 14 wherein the optical detector array includes an array of avalanche photodiodes.

16. The functional NIRS system of claim 14 wherein the optical detector array is a multi-anode photomultiplier tube.

17. The functional NIRS system of claim 14 wherein the optical detector array is a CMOS imaging device.

18. The functional NIRS system of claim 14 wherein the optical detector array is a charge-coupled imaging device.

19. The functional NIRS system of claim 1 wherein the second optical fiber bundle terminates on the inside surface of the elastomeric cap in a packed hexagonal pattern with each receive optical fiber in the second optical fiber bundle located at a vertex of a hexagon.

20. The functional NIRS system of claim 19 wherein each optical fiber in the first optical fiber bundle terminates on the inside surface of the elastomeric cap near the center of each hexagon in the packed hexagonal pattern.

21. The functional NIRS system of claim 20 wherein each hexagon in the packed hexagonal pattern has a characteristic size of about 2 millimeters.

22. The functional NIRS system of claim 1 wherein the first optical fiber bundle comprises about 4000 optical fibers and the second optical fiber bundle comprises about 8500 optical fibers and the elastomeric cap is approximately the size of a human scalp.

23. The functional NIRS system of claim 1 further comprising a set of calibration data stored in the first memory including an index of positions for the plurality of probe light beams, the position of each optical fiber in the first optical fiber bundle at the entrance plane, the position of each optical fiber in the second optical fiber bundle at the exit plane, an index of the terminated position of each optical fiber in the first optical fiber bundle as terminated on the elastomeric cap, an index of the terminated position of each optical fiber in the second optical fiber bundle as terminated on the elastomeric cap and an index relating each optical fiber in the second optical fiber bundle to a detector position in the optical detection system.

24. The functional NIRS system of claim 3 further comprising a data table stored in the first memory including:
an indexed assignment of each pseudo-orthogonal code in the set of pseudo-orthogonal codes to each optical fiber in the first optical fiber bundle; and,
the lateral positions of the each optical fiber as terminated on the elastomeric cap.

25. The functional NIRS system of claim 24 wherein the indexed assignment of the set of pseudo-orthogonal codes includes a strict level of orthogonality between optical fibers in the first optical fiber bundle that are terminated in close proximity at the elastomeric cap and further includes a reduced level of orthogonality between a second set of optical fibers and a third set of optical fibers, in the first optical fiber bundle, wherein the second set of optical fibers terminates near the center of the elastomeric cap and the third set of optical fibers terminates near the edge of the elastomeric cap.

26. (canceled)

27. (canceled)

28. A functional NIRS imaging system for hemodynamic imaging of subcutaneous tissue, incorporating a computer comprising a first processor, a memory, a persistent storage device and a display, comprising:
an elastomeric cap;
an optical fiber bundle, terminating together at a first end plane and terminating dispersively on the inside surface of the elastomeric cap, comprising a set of transmit optical fibers and a set of receive optical fibers;
a first light source producing a first light beam at a first wavelength and controlled by the computer;
a second light source producing a second light beam at a second wavelength and controlled by the computer;
an optical combiner combining the first light beam and the second light beam into a third light beam;
an optical collimator collimating the third light beam into a fourth light beam;
a modulation controller including a second processor and a second memory;
an optical modulation system, controlled by the modulation controller, which accepts the fourth light beam, converts the fourth light beam into a plurality of probe light beams, independently modulates each probe light beam in the plurality of probe light beams, programmatically directs each probe light beam in the plurality of probe light beams into a transmit optical fiber in the set of transmit optical fibers, and programmatically directs a plurality of collected light beams from the set of receive optical fibers into an optical detection system;
the optical detection system controlled by the first processor and connected to the first memory, which accepts the plurality of collected light beams from the optical modulation system, converts the plurality of collected light beams into a time series of electronic images and stores the time series of electronic images into the memory;
a computer readable media including a first set of programmed instructions, when executed by the first processor, converts the stored time series of electronic images into a set of hemoglobin oxygen saturation level maps, programmatically displays the set of hemoglobin oxygen saturation level maps on the display and stores the set of hemoglobin oxygen saturation level maps in the persistent storage device.

29. The functional NIRS system of claim 28 wherein the first set of program instructions, when executed by the first processor, controls the first light source and the second light source to alternate the wavelength of the fourth light beam between the first wavelength and the second wavelength.

30. The functional NIRS system of claim 29 further comprising a set of pseudo-orthogonal codes, of a predefined code length c, stored in the first memory and the second memory, each pseudo-orthogonal code corresponding to each transmit optical fiber in the optical fiber bundle.

31. The functional NIRS system of claim 30 wherein the computer readable media includes a second set of program instructions, when executed by the second processor, modulates the plurality of probe light beams with the set of pseudo-orthogonal codes.

32. The functional NIRS system of claim 30 wherein each probe light beam in the plurality of probe light beams is modulated by a pseudo-orthogonal code in the set of pseudo-orthogonal codes.

33. The functional NIRS system of claim 32 comprising:
a first set of c raw images stored by the optical detection system in the first memory and corresponding to the fourth light beam having the first wavelength; and,
a second set of c raw images stored by the optical detection system in the first memory and corresponding to the fourth light beam having the second wavelength.

34. The functional NIRS system of claim 33 wherein the first fiber bundle includes f fibers, further comprising:
a first set off intermediate images derived from the first set of c raw images and the set of pseudo-orthogonal codes; and,
a second set off intermediate images derived from the second set of c raw images and the set of pseudo-orthogonal codes.

35. The functional NIRS system of claim 34 further comprising:
a first resultant image stored in the first memory and derived from the first set of f intermediate images and a transformation matrix; and,
a second resultant image stored in the first memory and derived from the second set off intermediate images and the transformation matrix.

36. The functional NIRS system of claim 35 comprising a hemoglobin saturation map stored in the persistent storage device and derived from the first resultant image and the second resultant image.

37. The functional NIRS system of claim 28 wherein the modulation controller is configured with programmable instructions that when executed by the second processor modulates the plurality of probe light beams according to a modulation scheme selected from the group consisting of time division multiple access modulation, frequency division multiple access modulation and code division multiple access modulation.

38. The functional NIRS system of claim 28 wherein the optical modulation system includes a spatial light modulator and an optical lens.

39. The functional NIRS system of claim 38 wherein the spatial light modulator is a MEMS digital mirror device.

40. The functional NIRS system of claim 38 wherein the spatial light modulator is a liquid crystal light modulator.

41. The functional NIRS system of claim 28 wherein the optical detection system includes an optical lens and an optical detector array.

42. The functional NIRS system of claim 41 wherein the optical detector array includes an array of avalanche photodiodes.

43. The functional NIRS system of claim 41 wherein the optical detector array is a multi-anode photomultiplier tube.

44. The functional NIRS system of claim 41 wherein the optical detector array is a CMOS imaging device.

45. The functional NIRS system of claim 41 wherein the optical detector array is a charge-coupled imaging device.

46. The functional NIRS system of claim 28 wherein the optical fiber bundle terminates dispersively on the inside surface of the elastomeric cap in a packed hexagonal pattern with each receive optical fiber in the optical fiber bundle located at a vertex of a hexagon and each transmit optical fiber in the optical fiber bundle located near the center of a hexagon.

47. The functional NIRS system of claim 46 wherein each hexagon in the packed hexagonal pattern has a characteristic size of about 2 millimeters.

48. The functional NIRS system of claim 28 wherein the optical fiber bundle comprises about 4000 transmit optical fibers, about 8500 receive optical fibers and the elastomeric cap is approximately the size of a human scalp.

49. The functional NIRS system of claim 28 further comprising a set of calibration data stored in the first memory including an index of positions for the plurality of probe light beams, the position of each optical fiber in the optical fiber bundle at the first end plane, an index of the terminated position of each optical fiber in the optical fiber bundle as terminated on the elastomeric cap, and an index relating each receive optical fiber in the optical fiber bundle to a detector position in the optical detection system.

50. The functional NIRS system of claim 30 further comprising a data table stored in the first memory including:
an indexed assignment of each pseudo-orthogonal code in the set of pseudo-orthogonal codes to each transmit optical fiber in the set of transmit optical fibers; and,
the lateral positions of the each transmit optical fiber as terminated on the elastomeric cap.

51. The functional NIRS system of claim 50 wherein the indexed assignment of the set of pseudo-orthogonal codes enforces strict levels of orthogonality between optical fibers in the set of transmit optical fibers terminated in close proximity at the elastomeric cap and further enforces reduced levels of orthogonality between a first set of transmit optical fibers, terminating near the center of the elastomeric cap and a second set of transmit optical fibers, terminating near the edge of the elastomeric cap.

52. A method for hemodynamic imaging of subcutaneous tissue utilizing a computer, with a first processor and a first memory, an optical modulator connected to a programmable modulation controller with a second processor and a second memory, and a detector array connected to the computer, the method comprising the steps of:
providing at least one optical fiber bundle including a set of transmit fibers terminating at the surface of an elastomeric cap and a set of receive fibers terminating at the surface of an elastomeric cap;
providing a collimated light beam;
alternating the wavelength of the collimated light beam between wavelengths L1 and L2;
dividing the collimated light beam into a set of probe beams in the optical modulator;
modulating the set of probe beams with the optical modulator using a set of modulation codes;
transmitting the set of probe beams through the set of transmit fibers and through the elastomeric cap wherein each probe beam is transmitted primarily by a single optical fiber;
collecting photons scattered from the subcutaneous tissue below the elastomeric cap into the set of receive fibers;
delivering the photons from the set of receive fibers to the detector array wherein each fiber in the at least one optical fiber bundle is imaged onto a subset of detectors in the detector array to form a set of image data;
processing the set of image data to create two high resolutions images of the subcutaneous tissue wherein the two high resolution images include a first high resolution image corresponding to the concentration of HbO and a second high resolution image corresponding to the concentration of Hb; and,
combining the two high resolution images together to create a hemoglobin oxygen saturation image of the subcutaneous tissue.

53. The method of claim 52 including the steps of:
providing a transmit optical fiber bundle for the set of transmit fibers;
providing a receive optical fiber bundle for the set of receive fibers;

54. The method of claim 52 including the steps of:
mapping each receive fiber in the set of receive fibers to each image pixel in the detector array corresponding to the each receive fiber's position on the elastomeric cap; and,
delivering the photons from the set of receive fibers to the detector array with the optical modulator according to the mapping.

55. The method of claim 52 including the steps of:
determining a fiber map P which maps each transmit fiber in the set of transmit fibers to a probe beam in the set of probe beams;
assigning the set of modulation codes M of code chip length c, and wherein one modulation code is assigned for each transmit fiber in the set of transmit fibers;
combining the fiber map P and the set of modulation codes M into a set of modulation matrices S; and,
storing the fiber map P, the set of modulation codes M and the set of modulation matrices S in the programmable modulation controller.

56. The method of claim 55 wherein the set of modulation matrices S is calculated according to:
$$S ( {t}_{n} ) = {\sum }_{i = 1}^{f} {P}_{i} \cdot {M}_{i} ( {t}_{n} )$$
where tₙ=n Δt, n=1 to the chip code length c, Δt is a predefined dwell time and where the index i ranges over the number of fibers f in the set of transmit fibers.

57. The method of claim 55 including the additional steps of:
configuring the collimated light beam with light at the wavelength L1;
modulating the set of probe beams with the set of modulation matrices S(tn) during time intervals to =n Δt, where n=1 to the chip code length c and Δt is a predefined dwell time;
reconfiguring the collimated light beam with light at the wavelength L2; and
repeating the step of modulating the set of probe beams with the modulation matrices S(tn).

58. The method of claim 57 including the steps of:
recording a set of raw images X1 in the detector array after step b and before step c during the time intervals t1 to tc;
recording a set of raw images X2 in the detector array after step d during the time intervals t1 to tc;
correlating the set of raw images X1 to the modulation codes M to form a first set of intermediate images R1;
correlating the set of raw images X2 to the modulation codes M to form a second set of intermediate images R2;
combining the first set of intermediate images into a cap oriented image for wavelength L1;
combining the second set of intermediate images into a cap oriented image for wavelength L2;
combining the cap oriented image for wavelength L1 with the cap oriented image for the wavelength L2 to arrive at a hemoglobin oxygen saturation image of the subcutaneous tissue.

59. The method of claim 58 wherein the steps of correlating the set of raw images X1 and correlating the set of raw images X2 to the set of modulation codes M is performed according to the formula:
$${R}_{λ k} = {\sum }_{n = 1}^{c} {X}_{λ} ( {t}_{n} ) \cdot {M}_{k} ( {t}_{n} )$$
where λ is an index ranging from 1 to 2 corresponding to the wavelengths L 1, L2; k is a fiber index ranging from 1 to the number of fibers f in the set of transmit fibers; n is an index ranging from 1 to the code chip length c; X_{λ} (tₙ) is the set of raw images at times tₙ for the wavelength L_{λ}; Mₖ(tₙ) is the nth modulation code chip for the kth fiber index and R_{λk} is an intermediate image for the kth fiber index and for wavelength L_{λ}.

60. The method of claim 59 wherein the steps of combining the first set of intermediate images and combining the second set of intermediate images is performed according to the formula:
F_{λk}=map(R_{λk})
where λ is an index ranging from 1 to 2 corresponding to the wavelengths L1, L2; k is a fiber index ranging from 1 to the number of fibers f in the set of transmit fibers; F_{λk} is the cap oriented image for the kth fiber index and wavelength L_{λ}, R_{λ}, is the set of intermediate images for L_{λ}, and the map function performs the steps of:
converting an input set of intermediate images to physical dimensions of the elastomeric cap; and,
mapping an image pixel to a position on the elastomeric cap.

61. The method of claim 60 wherein the step of combining the cap oriented image for wavelength L1 with the cap oriented image for the wavelength L2 to arrive at a hemoglobin oxygen saturation image of the subcutaneous tissue includes the steps of:
creating a transformation matrix A that incorporates a model of physical transformations for improving image resolution and for applying a set of instrument calibrations including optical fiber losses in the at least one optical fiber bundle;
applying the transformation matrix A according to the formula:
Iₕ=AF_{λk}
where λ is an index ranging from 1 to 2 corresponding to the wavelengths L1, L2; k is a fiber index ranging from 1 to the number of fibers f in the transmit optical fiber bundle; F_{λk} is the cap oriented image for the kth fiber index and wavelength L_{λ}; Iₕ includes a high resolution image for HbO concentration and a high resolution image for Hb concentration;
determining a hemoglobin oxygen saturation image by calculating a ratio of HbO concentration to total (HbO+Hb) concentrations for each pixel in Iₕ to arrive at the hemoglobin oxygen saturation image of the subcutaneous tissue.

62. The method of claim 61 wherein the step of creating the transformation matrix A includes incorporating reconstruction techniques to de-noise the first and second sets of intermediate images.

63. The method of claim 62 wherein the step of incorporating reconstruction techniques to de-noise the first and second sets of intermediate images includes selecting a reconstruction method from the group consisting of: applying lower-level regularization in a Moore-Penrose inverse transformation, applying deblurring techniques based on Bayesian priors, applying synthetic aperture analysis and applying any combination thereof.

64. The method of claim 61 wherein the step of applying the transformation matrix A is repeated to create a set of image pairs Iₕ (d) for varying depths d and the step of determining hemoglobin oxygen saturation image is repeated to determine a three-dimensional hemoglobin oxygen saturation image.

65. The method of claim 52 wherein the step of combining the two high resolution images together to create a hemoglobin oxygen saturation image of the subcutaneous tissue includes selecting an image reconstruction method from the group consisting of: applying lower-level regularization in a Moore-Penrose inverse transformation, applying deblurring techniques based on Bayesian priors, applying synthetic aperture analysis and applying any combination thereof.

66. The method of claim 52 including the steps of:
providing a set of pseudo-orthogonal codes as the set of modulation codes; and,
assigning the set of pseudo-orthogonal codes to the set of transmit fibers based on the potential for optical crosstalk.

67. The method of claim 52 including the steps of:
providing a set of pseudo-orthogonal codes as the set of modulation codes wherein a pseudo-orthogonal code is assigned to each transmit fiber in the set of transmit fibers;
requiring strict orthogonality between pairs of pseudo-orthogonal codes in the set of pseudo-orthogonal codes assigned to pairs of transmit fibers in the set of transmit fibers, wherein the pairs of transmit fibers terminate in close proximity to one another on the elastomeric cap;
reducing the code chip length of the set of pseudo-orthogonal codes by reducing the level of orthogonality between a first set of transmit fibers in the at least one optical fiber bundle, terminating near the center of the elastomeric cap and a second set of transmit fibers in the at least one optical fiber bundle, terminating near the edge of the elastomeric cap.

68. The method of claim 52 including the steps of:
providing a set of pseudo-orthogonal codes as the set of modulation codes wherein a pseudo-orthogonal code is assigned to each transmit fiber in the set of transmit fibers;
assigning the same level of orthogonality to each pseudo-orthogonal code in the set of pseudo-orthogonal codes based on a set of cross-correlation coefficients.
